(19) United States
(12) Patent Application Publication
(10) Pub. No.: US 2004/0136926 A1
(43) Pub. Date: Jul. 15, 2004

(54) Intra-oral drug delivery system

(76) Inventors: Antony Raj Periathamby, Brookfield, WI (US); Andrew R. Dentino, Brookfield, WI (US)

Correspondence Address: QUARLES & BRADY LLP, 411 E. WISCONSIN AVENUE, SUITE 2040, MILWAUKEE, WI, 53202-4497, US

(21) Appl. No.: 10/704,171

(22) Filed: Nov. 6, 2003

Related U.S. Application Data
(60) Provisional application No. 60/424,990, filed on Nov. 8, 2002.

(51) Int. Cl. A61K 7/28; C12N 9/00

(52) U.S. Cl. 424/50; 435/183

(57) ABSTRACT

A bifunctional peptide molecule is disclosed. In one embodiment, the molecule comprises at least two segments, wherein the first segment comprises a tooth enamel binding region of a salivary protein and wherein the second segment comprises an antimicrobial domain of a naturally occurring antimicrobial polypeptide, wherein the first segment and the second segment are linked by a biodegradable bond.

### CROSS-REFERENCE TO RELATED APPLICATION

The application claims priority to provisional patent application No. 60/424,990, filed Nov. 8, 2002, incorporated by reference herein.

### STATEMENT REGARDING FEDERALLY SPONSORED RESEARCH OR DEVELOPMENT

This invention was made with United States Government support awarded by the following agency: USPHS IR21DEO134565. The United States has certain rights in this invention.

### BACKGROUND OF THE INVENTION

Caries and periodontal disease remain the most common infections in humans, despite the use of antibiotics, fluorides and other oral health agents. Antibiotic resistance acquired by oral microorganisms amplified the need to develop new and innovative strategies for controlling plaque-related oral infections. One objective of our invention, described below, is to provide a new, effective, novel and non-toxic molecule for the prevention and treatment of plaque-related oral infections.

A. Salivary Proteins. The selective adsorption of salivary proteins from the oral environment forms the acquired enamel pellicle that covers human tooth surfaces. These salivary molecules include mucins, α-amylase, proline-rich glycoprotein (PRG), proline-rich proteins (PRPs), cystatins, statherin, and histatins. Some of these salivary molecules, present in the acquired pellicle, serve as receptors for bacterial colonization (Amano, et al., Infect. Immun. 62:3372-3380, 1994; Amano, et al., Infect. Immun. 64:1631-1637,1996; Amano, et al., Infect. Immun. 64:4249-4254, 1996a). Structural studies on salivary PRPs, PRG, and statherin have revealed the structural requirements for their attachment to the tooth enamel, and the functional domain that promotes bacterial attachment to the tooth surface (Amano, et al., supra, 1996; Amano, et al., supra, 1996a). Salivary statherin, the phosphorylated polypeptide, has been shown to have high affinity for calcium phosphate minerals. The amino-terminal 15-residue fragment (SN15) and its analog (SN_{A}15) exhibit higher affinity for HAP than the whole molecule. The C-terminal 15 residue fragment SC15 has been shown to mediate bacterial attachment to apatitic surfaces (Raj, et al., J. Biol. Chem. 267:5968-5676, 1992; Amano, et al., supra, 1996; Amano, et al., supra, 1996a). More importantly, the salivary proteins, such as histatins, statherin and mucins that form the acquired enamel pellicle and promote bacterial colonization, also have regions that exhibit antimicrobial activity. These inherently present antimicrobial sequences appear to be proteolytically degraded in saliva and regulate the bacterial colonization on the tooth surface (Antonyraj, et al., Arch. Biochem. Biophys. 356:197-208, 1998; Raj and Antonyraj, J. Dent. Res. 80:629, 2001).

B. Naturally Occurring Antimicrobial Polypeptides. Bactenecins, defensins and histatins in human saliva as well as defensins and bactenecins in neutrophils play a significant role in host defense. Both bactenecins and defensins exhibit broad-spectrum antimicrobial efficacy at micromolar concentrations against Gram-positive and Gram-negative bacterial, fungi and viruses (Selsted and Quellefte, Trends Cell Biol. 5:114-119, 1995; Raj and Edgerton, FEBS Let. 368:526-530, 1995; Raj, et al., supra, 1996; Raj, et al., Int. J. Oral Biol. 22:73-80, 1997). Fragments of defensins with 12 to 16 residues, containing one disulfide bridge, that mimic the three dimensional structure of intact molecules, exhibit microbicidal activity against oral pathogens such as C. albicans, A. actinomycetemcomitans, P. gingivalis and S. mutans. The potential application of defensin-like small peptides as dental therapeutics has been emphasized (Miyasaki and Lehrer, Agents 9:269-280, 1998; Raj, et al., Biochem. J. 347:633-641, 2000; Raj, et al., Biopolymers 53:281-292, 2000a). The amphiphilic structure of bactenecin and defensin fragments appears to facilitate spontaneous insertion into microbial membranes causing cell death (Raj et al., supra, 1996; Raj, et al., supra, 2000; Raj, et al., supra, 2000a). Defensins and bactenecins interact with lipopolysaccharides in Gram-negative bacteria, polysaccharides (teichoic acid) in Gram-positive bacteria, and phospholipids (phosphotidyl-glycerol).

Because these membrane components are not found in mammalian cells, the molecules are electrostatically specific for prokaryotic cells (Weinberg, et al Crit. Rev. Oral Biol. Med. 9:399-414, 1998). Normal gingival epithelial cells express β-defensins (Krisanaprakornkit, et al., Infect. Immun. 66:4222-4228, 1998) suggesting their importance in host-pathogen interaction at the oral mucosal barrier and their relevance in oral health. Impairment of β-defensin activity could cause chronic bacterial infections in cystic fibrosis patients (Smith, et al., Cell 85:229-239, 1996; Goldman, et al., Cell 88:553-560, 1997). P. gingivalis strongly inhibits the accumulation of interleukin 8 by gingival epithelial cells in response to components of normal oral flora (Darveau, et al., Infect. Immun. 66:1660-1665, 1998). These studies indicate that oral pathogens might circumvent natural defense mechanisms and emphasize the need to develop new methods of delivering natural antimicrobial agents.

C. Local Delivery of Drugs into the Oral Cavity. Methods employed to deliver the antimicrobial agents into the site of oral infection include rinsing, irrigation, systemic administration, and local application using sustained and controlled delivery devices (Greenstein and Polson, J. Periodontal. 69:507-520, 1998). The efficiency of the drug delivery system designed to target oral infections depends on its ability to deliver the drug at a bacteriostatic or bactericidal concentration and its ability to retain the medicament long enough to ensure adequate results. Since local drug delivery can achieve these requirements, commercially available formulations such as tetracycline fibers, metronidazole and minocycline gels, chlorhexidine chips, and doxycycline polymers have recently been approved by the Food and Drug Administration (Greenstein and Polson, supra, 1998). In these systems, polymers encapsulating an antibiotic or antiseptic, when inserted or injected into the site of infection, degrade and release the encapsulated drug, maintaining adequate drug concentration at the site of infection. The major limitation of the currently available local drug delivery systems include the following: (i) adverse events such as dental or gingival pain, soreness, discomfort, and sensitivity; (ii) difficulty in inserting or injecting the drug delivery device around all teeth; (iii) difficulty in retention of the device at the tooth surface or pockets for a sufficient length of time; and (iv) toxic effects and bacterial resistance induced by the conventional antibiotics used in the devices.

In contrast to the existing formulations that enclose conventional antibiotics within a biodegradable polymer that has the least affinity for the tooth or pellicle surface, our invention, described below, teaches a method of linking a safe, natural antimicrobial agent with an enamel binding peptide using biodegradable bonds. The resulting molecules will adhere to the tooth and pellicle surfaces uniformly, thereby inhibiting microbial accumulation that leads to plaque formation.

### BRIEF SUMMARY OF THE INVENTION

To prevent and control oral and plaque-related infections, it is important to have a drug delivery system that targets the tooth surface. Equally important is that the molecules to be developed have minimal chances of inducing drug resistance. The present invention provides bifunctional hybrid molecules useful in an efficient intraoral delivery system that will: (1) provide a sustained release of an antimicrobial agent due to its high affinity for the tooth surface; (2) hinder the binding sites on tooth and pellicle surfaces to inhibit bacterial re-colonization promoted by the adsorbed salivary proteins; (3) disrupt bacterial cell membranes leading to cell death due to the presence of the selective natural antimicrobial agent, thereby reducing the chances of inducing drug resistance among oral pathogens, and (4) eliminate the discomforts and other disadvantages of the currently available drug delivery devices. The proposed drug delivery system will be more efficient than the media encapsulated forms for targeted distribution of antimicrobial agents. Unlike the other local delivery devices that require professional insertion or injection, the molecules can be easily applied and replenished by rinsing, brushing, or irrigation. They could also be used professionally for site-specific applications to sub-gingival areas, if required. The kinetics of the antibiotic release can be altered by suitable modification of the biodegradable linkage. This system also will eliminate the sensitivity problems experienced with the insertion or injection of drug delivery devices.

The present invention provides hybrid molecules by rationally connecting a tooth enamel binding region, preferably from salivary proteins, and an antimicrobial domain, preferably from bactenecins or defensins. This invention provides dental therapeutics that could supplement and replace mechanical methods for controlling plaque and associated oral infections. The function of native antimicrobial agents, or their analogs in the form of a bifunctional hybrid molecule targeting the tooth surface, mimics the salivary defense mechanism. The hybrid molecules would strengthen and replenish the natural defense mechanisms hampered by oral pathogens during oral infections especially when the host is under stress.

In the salivary mucin glycoprotein MG2 sequence, the six tandem repeats represented by the sequence T-T-A-A-P-P-T-S-A-T-T-P-A-P-P-S-S-S-A-P-P-E have been reported to elicit antimicrobial activity against oral pathogens (Antonyraj, et al., supra, 1998), indicating the inherently present antimicrobial regions. In addition, the N-terminal 30 residue fragment of proline-rich protein (PRP-1) has been reported to be the tooth enamel binding domain. The C-terminal segment which is rich in proline, glutamine and glycine has been shown to serve as a receptor for microbial adherence on the tooth surface (Gibbons and Hay, Infect. Immun. 56:439-445, 1988; Gibbons, et al., Infect. Immun. 59(9):2948-2954, 1991).

Studies on the structural biology of bactenecins and defensins have identified regions BN16 and DN13, respectively, which mimic the structure and microbicidal potency of the native molecules (Raj, et al., supra, 1996; Raj, et al., Int. J. Oral Biol. 22:73-80, 1997; Raj, et al., supra, 2000; Raj, et al., supra, 2000a; Appendix Ms. 6-9; Raj and Genco, 1996).

In one embodiment, the present invention is a bifunctional peptide molecule comprising at least two segments, wherein the first segment comprises a tooth enamel binding region of a salivary protein and wherein the second segment comprises an antimicrobial domain of a naturally occurring antimicrobial polypeptide, wherein the first segment and the second segment are linked by a biodegradable bond. The first segment is preferably a fragment of a salivary protein selected from the group consisting of mucins, α-amylase, proline-rich glycoprotein (PRG), proline-rich proteins (PRPs), cystatins, statherin, and histatins. Most preferable are statherin fragments. The naturally occurring antimicrobial polypeptide is preferably a defensin, a histatin, or a bactenecin.

In another embodiment, the invention is a method of intra-orally delivering antimicrobial agents to tooth or pellicle surfaces, comprising the step of administering an effective amount of the molecule described above to the tooth or pellicle surface.

In another embodiment, the present invention is a bifunctional peptide molecule comprising at least two segments wherein the first segment comprises a tooth enamel binding region of a salivary protein and wherein the second segment comprises a therapeutic molecule selected from the group consisting of anti-inflammatory agents, immuno-modulators, growth factors, and biofilm signaling agents and inhibitors; wherein the first and second segments are linked by a biodegradable bond.

### BRIEF DESCRIPTION OF THE SEVERAL VIEWS OF THE DRAWINGS

FIG. 1 is a diagram of suitable carrier peptides (tooth enamel binding region), biodegradable bonds, and antimicrobial agents of the present invention.

FIG. 2 is a synthetic scheme employed for a typical synthesis of anhydride and ester bonds.

FIG. 3 is a schematic representation of an ester-linked bifunctional molecule wherein serine side chain is used to link the carrier and the antimicrobial domains.

FIG. 4 is a graph of antimicrobial peptide release in human clarified whole saliva.

FIG. 5 is a graph of candidacidal activity as a function of the antimicrobial peptide release. The data at −5° C. indicates the activity of whole hybrid.

FIG. 6 is a graph demonstrating peptide adsorption to hydroxyapatite beads.

FIG. 7 is a graph demonstrating candidacidal activity of the hybrid adsorbed HAP surface as compared to the control HAP surface.

FIG. 8 is a graph demonstrating bactericidal activity of hybrid adsorbed HAP surface as compared to the control HAP surface.

### DETAILED DESCRIPTION OF THE INVENTION

1. In General

Caries and periodontal disease remain the most common infections in humans, despite the use of antibiotics, fluorides and other oral health agents. Antibiotic resistance acquired by oral microorganisms amplifies the need to develop new and innovative strategies for controlling plaque-related oral infections. The present invention pertains to new and novel bifunctional molecules capable of intra-orally delivering antimicrobial agents and other drugs to the tooth and pellicle surfaces, preferably used in the form of a mouth rinse, toothpaste or applied as a subgingival irrigant. These hybrid molecules can be used for treating periodontal and other oral infectious diseases that require a sustained release of antimicrobial and other drug agents from the tooth surface and to prevent microbial colonization leading to gingivitis, caries and plaque formation.

In one embodiment of the present invention, we utilize the apatitic/tooth surface binding region of a salivary protein, such as statherin, as a carrier molecule for naturally-occurring antimicrobial polypeptides. In salivary defense molecules, the carrier components are connected to antimicrobial sequences by secondary amide (peptide) bonds. The cleavage and release of the antimicrobial sequences from salivary defense proteins require healthy physiological and cellular functions for specific proteolysis. The strategy of the present invention is to design bifunctional molecules wherein the carrier and the antimicrobial sequences are linked by a variety of biodegradable bonds (such as anhydride, ester, peroxide, ether or disulfide bond) to facilitate a differential degradation and sustained release of the antimicrobial or the drug including anti-inflammatory agents, immuno-modulators and inhibitors of biofilm formation.

2. Local Delivery of Drugs into the Oral Cavity

a. Currently Available Methods.

To prevent and control oral and plaque-related infections, it is an advantage to have a drug delivery system that targets the tooth surface. Equally important is that the molecules have minimal chances of inducing microbial drug resistance. Currently available methods to deliver antimicrobial agents into the site of oral infection include rinsing, irrigation, systemic administration, and local application using sustained and controlled delivery devices (Greenstein and Poison, supra, 1998). The efficiency of the drug delivery system designed to target oral infections depends on its ability to deliver the drug at a bacteriostatic or bactericidal concentration and its ability to retain the medicament long enough to ensure adequate results.

Because local drug delivery can achieve these requirements, commercially available formulations such as tetracycline fibers, metronidazole and minocycline gels, chlorhexidine chips, and doxycycline polymers have recently been approved by the Food and Drug Administration (Greenstein and Poison, supra, 1998). In these systems, polymers encapsulating an antibiotic or antiseptic, when inserted or injected into the site of infection, degrade and release the encapsulated drug, maintaining adequate drug concentration at the site of infection.

The major limitations of the currently available local drug delivery systems include the following: i) adverse events such as dental or gingival pain, soreness, discomfort, and sensitivity; ii) difficulty in inserting or injecting the drug delivery device around all teeth; iii) difficulty in retaining the device at the tooth surface or pockets for a sufficient length of time; iv) toxic effects and bacterial resistance induced by conventional antibiotics used in such delivery devices and v) applied professionally by a dentist and expensive. Moreover, the existing delivery devices generally utilize bio-degradable polymers and conventional organic antibiotics.

b. Salivary Proteins as Drug Carriers.

The selective adsorption of salivary proteins from the oral environment forms the acquired enamel pellicle that covers human tooth surfaces. Exemplary salivary proteins include mucins, α-amylase, proline-rich glycoprotein (PRG), proline-rich proteins (PRPs), cystatins, statherin, and histatins. We envision that one would isolate and/or replicate the tooth enamel binding region of a salivary protein for use in the present invention. The present invention utilizes the extensive studies carried out on salivary statherin and its functionally active region for binding to the tooth enamel surface (Raj, et al., J. Biol. Chem. 267:5968-5976, 1992). By “tooth enamel binding region” we mean a portion of the protein sequence that has a natural and highly specific binding affinity for the tooth surface. As discussed below, this portion may comprise only the residues with the highest specific binding affinity for the tooth surface or may comprise a larger region. Additionally, one may make innocuous or functionally insignificant changes to the naturally occurring amino acid sequence and still be within the “tooth enamel binding region” of the present invention. The peptide sequence and the structural requirements for binding to the tooth enamel surface have been identified by using fragments of salivary statherin and their analogs (Raj, et al., supra, 1992). The protein fragments and analogs are preferably synthesized by using solid-phase procedures.

In this invention, one would preferably use only the tooth enamel binding region of salivary proteins as the carrier component of the bifunctional hybrid molecule. However, including other regions of the salivary molecule will not diminish or significantly enhance the binding efficacy of the carrier component. It is important not to include the region that promotes microbial adherence to the tooth enamel surface. Adding additional residues/regions might increase the cost of production with no clinical significance.

Some of these salivary molecules, present in the acquired enamel pellicle, serve as receptors for bacterial colonization (Amano, et al., supra, 1994; Amano, et al., supra, 1996; Amano, et al., supra, 1996a). Structural studies on salivary PRPs, PRG, and statherin have revealed the structural requirements for their attachment to the tooth enamel (Raj, et al., supra, 1992), and the functional domain that promotes bacterial attachment to the tooth surface (Amano, et al., supra, 1996; Amano, et al., supra, 1996a). Salivary statherin, the phosphorylated polypeptide, has been shown to have high affinity for calcium phosphate minerals. The amino-terminal 15-residue fragment (SN15) and its analog (SN_{A}15) exhibit higher affinity for HAP than the whole molecule. The C-terminal 15 residue fragment SC15 has been shown to mediate bacterial attachment to apatitic surfaces (Raj, et al., supra, 1992; Amano, et al., supra, 1996; Amano, et al., supra, 1996a).

More importantly, the salivary proteins, such as histatins, statherin and mucins that form the acquired enamel pellicle and promote bacterial colonization, also have regions that exhibit antimicrobial activity. These inherently present antimicrobial sequences appear to be proteolytically degraded in saliva and regulate the bacterial colonization on the tooth surface (Antonyraj, et al., supra, 1998; Raj and Antonyraj, supra, 2001). The identification of these three distinct functional domains for apatitic/tooth surface binding, microbial adherence to the tooth surface, and antimicrobial activity in salivary proteins, are important in our design of molecules that could possess high affinity for the tooth enamel surface and antimicrobial activity.

The functional domains in salivary proteins are typically identified by synthesizing various regions in the molecule, and assessing their properties as compared to those of the intact molecules (Raj, et al., supra, 1992; Amano, et al., supra, 1996). Moreover, the structural requirements for these functions are determined by rationally designing analogs of these regions. The tooth enamel binding domain of salivary statherin showed an α-helical model where in the negative charges are separated in space by 4.6-6.2 Å closely matching the interatomic distance between calcium ions on the (0 0 1 ) face of the hydroxyapatite crystal, thereby accounting for the affinity of this molecule (Raj, et al., supra, 1992). Such studies provide a rationale and information to derive peptide sequences that could efficiently bind to the tooth enamel.

c. Naturally Occurring Antimicrobial Polypeptides.

Defensins and histatins in human saliva as well as defensins and bactenecins in neutrophils play a significant role in host defense. Both bactenecins and defensins exhibit broad-spectrum antimicrobial efficacy at micromolar concentrations against Gram-positive and Gram-negative bacteria, fungi and viruses (Selsted and Quellette, supra, 1995; Raj and Edgerton, supra, 1995; Raj, et al., supra, 1996; Raj, et al., supra, 1997). Fragments of defensins with 12 to 16 residues, containing one disulfide bridge, that mimic the three dimensional structure of intact molecules, exhibit microbicidal activity against oral pathogens such as C. albicans, A. actinomycetemcomitans, P. gingivalis and S. mutans.

The potential application of defensin-like small peptides as dental therapeutics has been discussed (Miyasaki and Lehrer, supra, 1998; Raj, et al., supra, 2000; Raj, et al., supra, 2000a). The amphiphilic structure of bactenecin and defensin fragments appears to facilitate spontaneous insertion into microbial membranes causing cell death (Raj, et al., supra, 1996; Raj, et al., supra, 2000; Raj, et al., supra, 2000a). Defensins and bactenecins interact with lipopolysaccharides in Gram-negative bacteria, polysaccharides (teichoic acid) in Gram-positive bacteria, and phospholipids (phosphotidyl-glycerol). Since these structures are not found in mammalian cells, the molecules are electrostatically specific for prokaryotic cells (Weinberg, et al., supra, 1998). Normal gingival epithelial cells express β-defensins (Krisanaprakornkit, et al., supra, 1998) suggesting their importance in host-pathogen interaction at the oral mucosal barrier and their relevance in oral health. Impairment of β-defensin activity could cause chronic bacterial infections in cystic fibrosis patients (Smith, et al., supra, 1996; Goldman, et al., supra, 1997). P. gingivalis strongly inhibits the accumulation of interleukin 8 by gingival epithelial cells in response to components of normal oral flora (Darveau, et al., supra, 1998).

In the present invention, we use fragments of natural bactenecins and defensins as antimicrobial components of the bifunctional intra-oral delivery molecules. By “antimicrobial domain of a naturally occurring antimicrobial polypeptide” we mean a functional region which elicits the microbial activity of the whole molecule. Structure-function studies establish the functional domains in large molecules. These domains are preferably synthesized by solid-phase procedures. One may typically wish to use the smallest peptide possible that contains microbial activity, but one may also wish, for convenience, to include additional residues on either side of this functional region. Additionally, one may wish to make innocuous or functionally irrelevant changes to the amino acid sequence. These peptides would still be within the “antimicrobial domain of a naturally occurring antimicrobial polypeptide” of the present invention.

3. Bifunctional Intra-Oral Delivery Molecules.

The present invention is the construction of hybrid peptide or molecule by rationally connecting the tooth enamel binding region of a salivary protein, preferably in salivary statherin, and the antimicrobial domain of a naturally occurring antimicrobial polypeptide, preferably bactenecins and defensins. The idea to construct such molecules stemmed from our knowledge of the structural biology of salivary proteins and natural antimicrobial bactenecins and defensins. Tables 3 and 4 provide a list of preferred efficient carrier molecules and antimicrobial agents that can be used in the synthesis of such hybrid molecules. Table 3 provides a list of efficient carrier molecules while Table 4 summarizes a list of antimicrobial peptides that represent fragments of defensins and bactenecins.

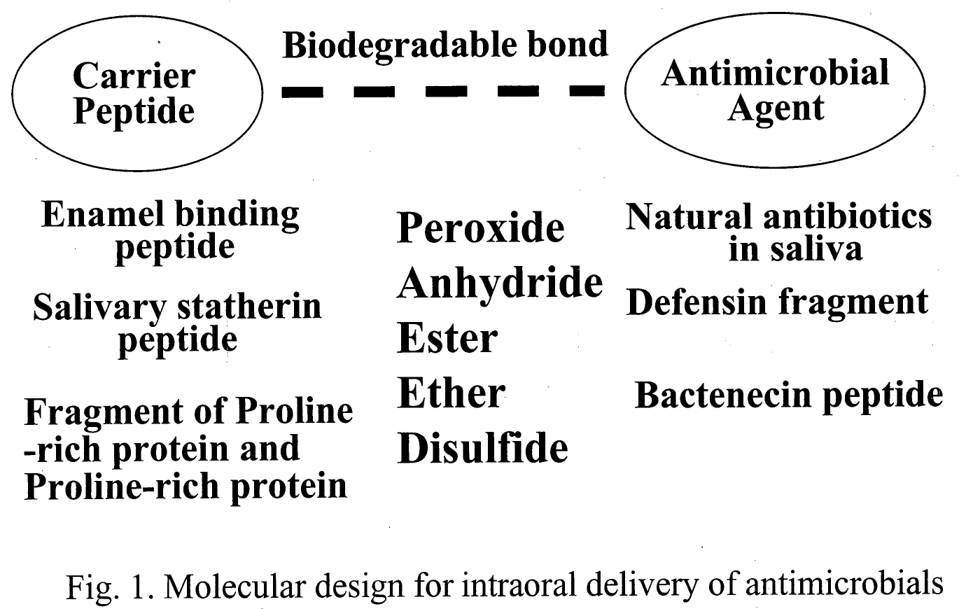

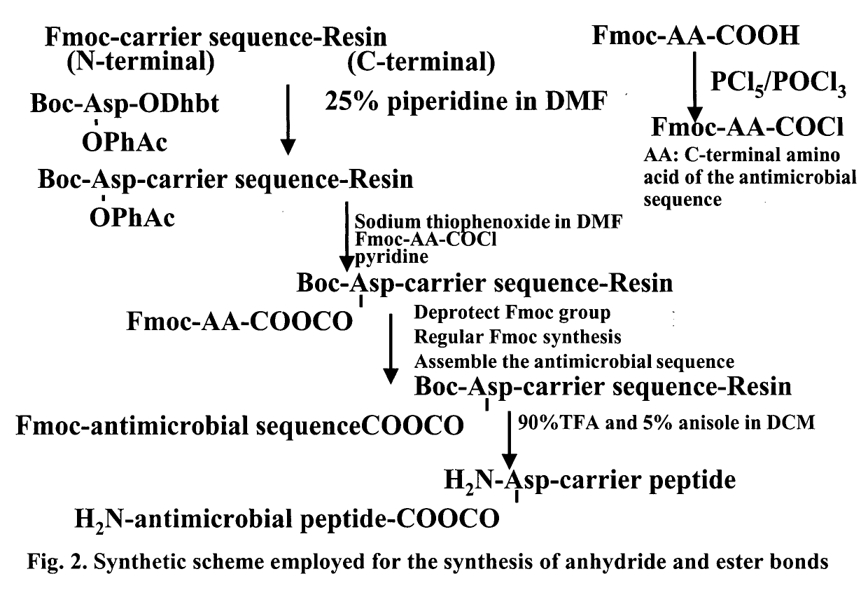

The design of such bifunctional molecules and the method of synthesis for a hybrid molecule containing the biodegradable ester and anhydride bonds are provided in FIG. 1 and FIG. 2, respectively.

In general, one would select a carrier molecule (tooth enamel binding region of a salivary protein) and antimicrobial domain and create a bifunctional molecule by using the following general method: The two preferred functional regions, namely binding to the tooth enamel surface and antimicrobial activity, are known and listed in Table 3 and Table 4. The respective sequences can be synthesized by standard conventional synthetic procedures. The use of different biodegradable bonds will vary the rate of release of the antimicrobial agent or the drug inherently present in the hybrid molecules. The differential rate of release of the therapeutic agent would have potential for clinical use for the treatment of oral infectious disease depending on the nature or intensity of the disease.

FIG. 2 discloses a particularly advantageous synthetic scheme employed for the synthesis of anhydride and ester bonds.

Referring to FIG. 2, the carrier sequence is to be assembled from the C-terminal using Wang resin (0.5 mmol/g) to which the C-terminal Fmoc amino acid is linked. After the last amino acid is coupled, as partic acid will be added to the resin using tBoc-aspartic acid β-phenacylester in the form of 3,4-dihydro-4oxo-1,2,3-benzotrizene (Dhbt) ester. The C-terminal amino acid of the antimicrobial sequence is converted to Fmoc-aminoacyl chloride by treatment with PCl₅ in the presence of POCl₃. The phenacyl protecting group on Asp residue in the peptidyl resin is deprotected with sodium thiophenoxide in dimethyl formamide and then treated with the Fmoc aminoacyl chloride. After this point, the antimicrobial chain is assembled using the standard Fmoc procedures. Phosphorylation of Ser residues and disulfide formation will be accomplished as reported previously (Raj, et al., supra, 1992; Raj, et al., supra, 2000a).

The procedure for an ester linkage is the same as described above except that the linker is serine, instead of aspartic acid. Briefly, after completion of the assembly of the carrier peptide using Fmoc procedures, tBoc-Ser is added via its Dhbt ester without protecting the side-chain OH group, since Dhbt esters specifically couple to amino function and not the OH groups. The C-terminal amino acid of the antimicrobial sequence is converted to Fmoc-aminoacyl chloride and coupled to the side-chain OH group of Ser residue to form the ester function. After this point, the antimicrobial sequence will be assembled.

The use of various biodegradable bonds to generate a variety of bifunctional molecules is important for different applications and clinical situations. The rate of release of the therapeutic molecule from the hybrid molecule varies with the degradable bonds. The pH of the human oral flora varies from acidic to neutral 6.5 to 7.2 and varies with individuals. In addition to pH, ionic concentration, microbial population, oxydising and reducing agents in saliva have marked influence on the rate of dissociation of these biodegradable bonds. In clinical situations, it may be necessary either to use a fast release or a slow release of the medication. Depending on the clinical situation, it may become necessary to use molecules that have a wide range in the rate of release of the pharmaceutical agents to the site of infection, injury and most importantly wound repair.

Esters and ethers may provide longer time release for slow later delivery of growth factors, and inhibitors of biofilm formation. Anhydride and peroxide linkages may provide a more potent short term and rapid release for immediate and early delivery of these molecules. Disulfide linkage could provide specific rate of release of antimicrobial agents in the presence of thiol-producing oral anaerobic bacterial pathogens. The rate of release in such cases will depend on the anaerobic bacterial content and the amount of thiol present in the oral environment.

The present invention is also the linkage of different therapeutic compounds, such as anti-inflammatory agents, immuno-modulators, growth factors or biofilm signalling agents or inhibitors such as the N-acyl-L-homoserine lactone to a carrier sequence. The carrier sequence and the synthetic scheme will allow biodegradable bonds so long as the pharmaceutical agent being delivered has functional groups such as carboxyl, hydroxyl, amino or sulfydryl group or by inserting such functional groups to the therapeutic molecules via synthesis.

For example, N-aceyl-L-homoserine lactone can be linked to the enamel binding component for the delivery of an inhibitor to prevent biofilm formation. Anti-histamine or glucocorticoids can be selectively linked to deliver anti-inflammatory agents. β-defensisns or its fragments can be used to promote dendritic and T cell activity. Zadaxin, interferon β1a, or interferon β1b can be attached to deliver immuno-modulators. Growth factors such as fibroblast growth factors can be linked to the enamel binding peptide for fibroblast cell proliferation leading to tissue growth.

4. Method of Intra-Oral Delivery of Antimicrobial Agents.

The bifunctional hybrid molecules could be added to mouthrinse, toothpaste or sugar-free gum. As a means of self-administering the therapeutic agent, they could also be professionally administered through sub-gingival irrigation applied at a dental clinic. Effective amounts of the agent will be dependent on determination of the potency of the therapeutic agent in the hybrid molecules well as the surface area of the tooth structure available for binding. One of skill could readily determine an effective range.

In one embodiment, the total concentration administered into the oral cavity is 100 μM (±10%). This concentration will not affect the oral ecology, since this concentration of natural antimicrobials is present in human saliva.

### EXAMPLES

The three distinct domains for the apatite/tooth surface binding, microbial adherence and antimicrobial activity in salivary proteins, have been well documented in literature. In the salivary statherin sequences shown below, these three functional regions have been identified, as reported previously (Raj, et al., supra, 1992; Appendix Ms.1: Amano, et al., supra, 1996; Amano, et al., supra, 1996a).

| | |
|---|---|
| | 5 10 15 20 25 30 35 40 43 |
| Statherin: | D-Sp-Sp-E-E-K-F-L-R-R-I-G-R-F-G-Y-G-Y-G-P-Y-Q-P-V-P-E-Q-P-L-Y-P-Q-P-Y-Q-P-Q-Y-Q-Q-Y-T-F |

Enamel binding region [1-15]: D-Sp-Sp-E-E-K-F-L-R-R-I-G-R-F-G,(Raj, et al., 1992)

Microbicidal region [6-21]: K-F-L-R-R-I-G-R-F-G-Y-G-Y-G-P-Y (Raj and Antonyraj, 2001)

Microbial adherence region [29-43]: L-Y-P-Q-P-Y-Q-P-Q-Y-Q-Q-Y-T-F (Amano et al., 1996; 1996a)

In addition, the N-terminal 30 residue fragment of proline-rich protein (PRP-1) has been reported to be the tooth enamel binding domain. The C-terminal segment, which is rich in proline, glutamine and glycine, has been shown to serve as a receptor for microbial adherence on the tooth surface (Gibbons and Hay, supra, 1988; Gibbons, et al., supra, 1991). Therefore, one may also use fragments of PRP and proline-rich glycoprotein as carrier components of the intra-oral delivery molecules.

Studies on the structural biology of bactenecins and defensins have identified regions BN16 and DN13, respectively, which mimic the structure and microbicidal potency of the native molecules (Raj, et al., supra, 1996; Raj, et al., supra, 1997; Raj, et al., supra, 2000; 2000a; Raj and Genco, supra, 1996).

[BN16:X-X-P-P-I-X-P-P-F-Y-P-P-F-X-P-P (X═R,BN16; X═K, BN16a, X=orninthine, BN16b; X=D-arginine, BN16c]

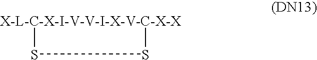

DN13 (X═R); DN12a (X═K)

DN13b (X=ornithine),

DN13c (X=D-arginine)

Organisms grown in saliva might be less susceptible to antibiotics due to the protective salivary film on their surface. At the same time, peptides in saliva may be degraded or inactivated by salivary and bacterial components. Hence, bactenecin and defensin analogs and each test organism were separately pre-incubated with human clarified whole saliva for 5 hours before the microbicidal assay was performed using C. albicans (DIS) and P. gingivalis (W50 and 381). The peptide stock solution prepared in 25% saliva buffer, pH 7.4 and 75% clarified whole saliva, was diluted with clarified whole saliva to obtain appropriate concentrations and maintained at 37° C. for 5 hours. The assay was performed by using the procedure described previously (Edgerton, et al., supra, 1998). Microbial strains were grown to early log phase, harvested, washed and re-suspended in clarified whole saliva to 10⁶ cells/ml. After 5 hours, 100 μl of peptide and 100 μl of cells were incubated for 90 minutes at 37° C. In the case of P. gingivalis, anaerobic conditions were maintained. The El₉₀ values (˜2-5 μM) provided in Table 1, below, are comparable to those observed in buffer medium (Raj, et al., supra, 1996; Raj, et al., supra, 1997; Raj, et al., supra, 2000). The data provide evidence for the stability and activity of bactenecin and defensin analogs in the presence of salivary and bacterial components. Thus, the data provide the rationale for selecting analogs of bactenecins and defensins as antimicrobial components to construct hybrid molecules.

TABLE 1
Microbicidal activity of bactenecins and defensin analogs after a 5 hour pre-incubation
of each agent and each pathogen separately with clarified whole saliva

| | C. albicans | P. gingivalis | | | C. albicans | P. gingivalis | |
|---|---|---|---|---|---|---|---|
| Definsin | DIS | W50 | 381 | Bactenecins | DIS | W50 | 381 |
| DN13 | 2.5 ± 0.6 | 2.5 ± 0.6 | 3.0 ± 0.4 | BN16 | 5.0 ± 1.5 | 5.0 ± 0.8 | 4.0 ± 0.6 |
| DN13a | 2.0 ± 0.2 | 2.0 ± 0.8 | 2.0 ± 0.5 | BN16a | 5.0 ± 1.4 | 4.0 ± 0.9 | 5.0 ± 0.4 |
| DN13b | 2.0 ± 0.8 | 2.5 ± 0.6 | 2.5 ± 0.6 | BN16c | 2.0 ± 0.5 | 1.9 ± 0.5 | 1.8 ± 0.9 |
| DN13c | 1.8 ± 0.6 | 2.0 ± 0.4 | 1.5 ± 0.4 | BN16c | 2.5 ± 0.4 | 2.2 ± 0.6 | 2.2 ± 0.4 |

Sensitivity is expressed as EI₉₀ (μm). Cells and peptide incubated for 1.5 hours at 37° C. and pH 7.4. Values are expressed as means ± SD from 3 independent experiments.

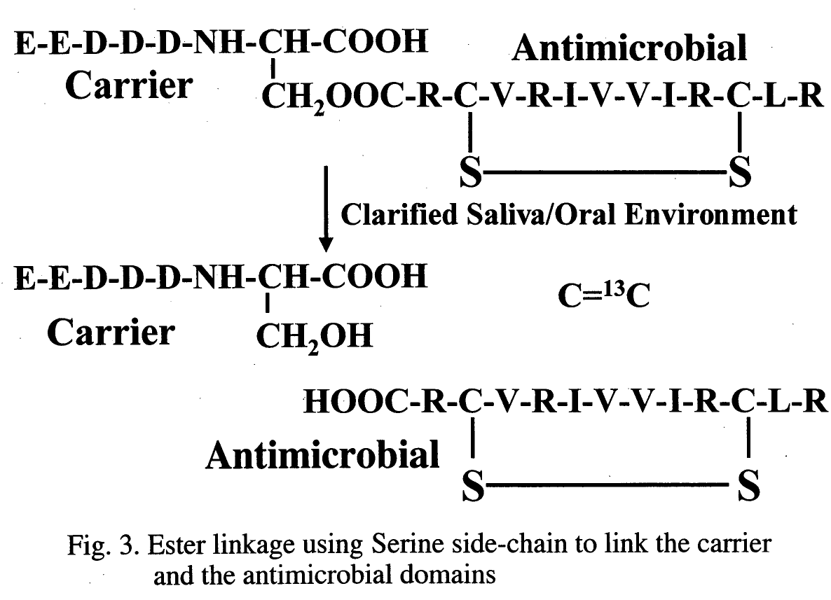

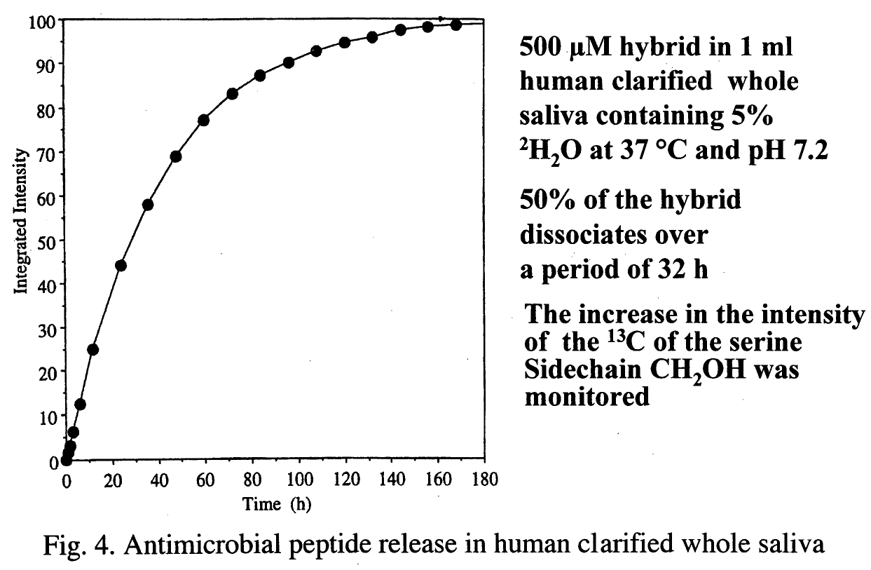

In order to determine the kinetics of release of the antimicrobial peptide in human clarified saliva, ¹³C-enriched serine amino acid was used to link its backbone with the carrier and its sidechain with the antimicrobial peptide as shown in FIG. 3. The ¹³C-NMR studies (500 μM of the hybrid in 1 ml clarified whole saliva) on the kinetics of the release of the antimicrobial agent in human clarified whole saliva, demonstrate a sustained release of the antimicrobial agent from the hybrid for a period of more than 60 hours, as described in FIG. 4. The in-vitro study indicates that such hybrid molecules in the salivary physiological environment might be stable and release the antimicrobial or the drug component for a prolonged period of time.

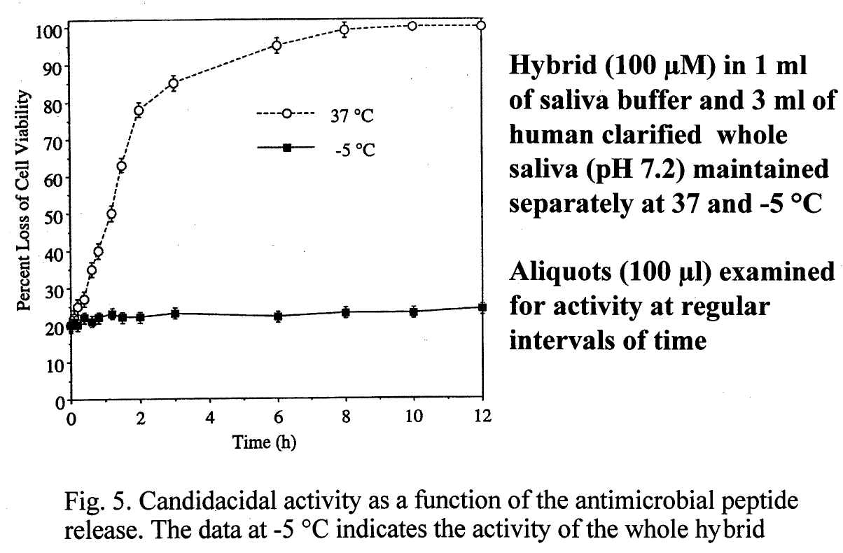

The fungicidal activity of the hybrid molecule was examined as a function of release of the antimicrobial agent using Candida albicans. [Hybrid (100 μM) in 1 ml of saliva buffer and 3 ml of human clarified whole saliva (pH 7.2) maintained separately at 37 and −5° C. Aliquots (100 μl) examined for activity at regular intervals of time]. The hybrid molecule itself induced 20% loss in cell viability as shown in the graph at −5° C. The release of the antimicrobial agent at 37° C. induced a pronounced fungicidal effect reaching 100% loss in cell viability within 6 hours as described in FIG. 5.

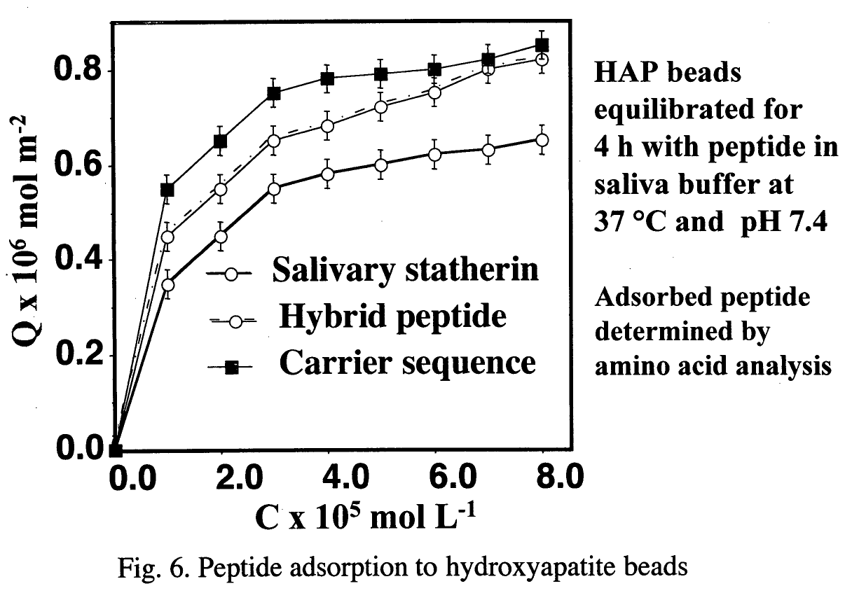

The extent to which the hybrid molecule gets adsorbed was examined using hydroxyapatite (HAP) beads as described previously (Raj, et al., supra, 1992). The affinity of the hybrid molecule for HAP was quite comparable to salivary statherin that has the greatest affinity for HAP mineral as compared with other salivary molecules. The affinity coefficients data are provided in FIG. 6.

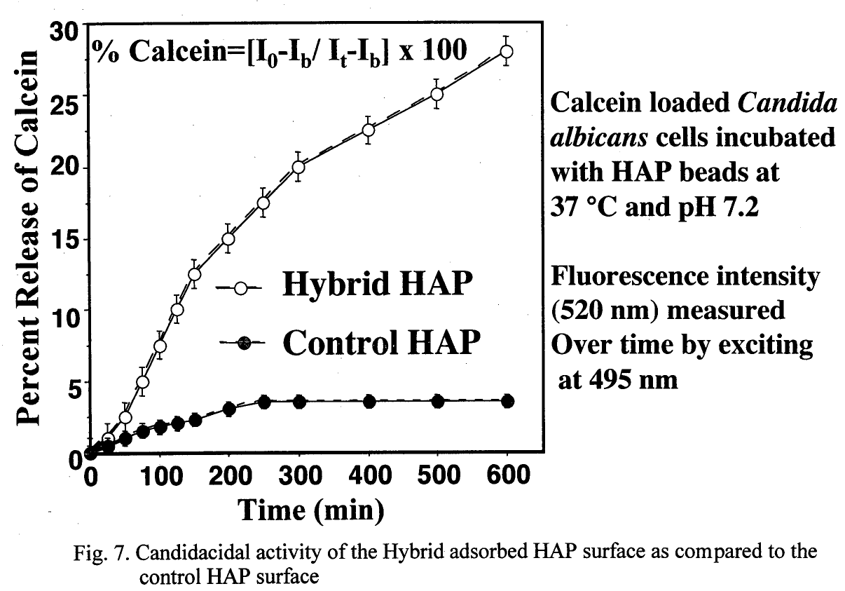

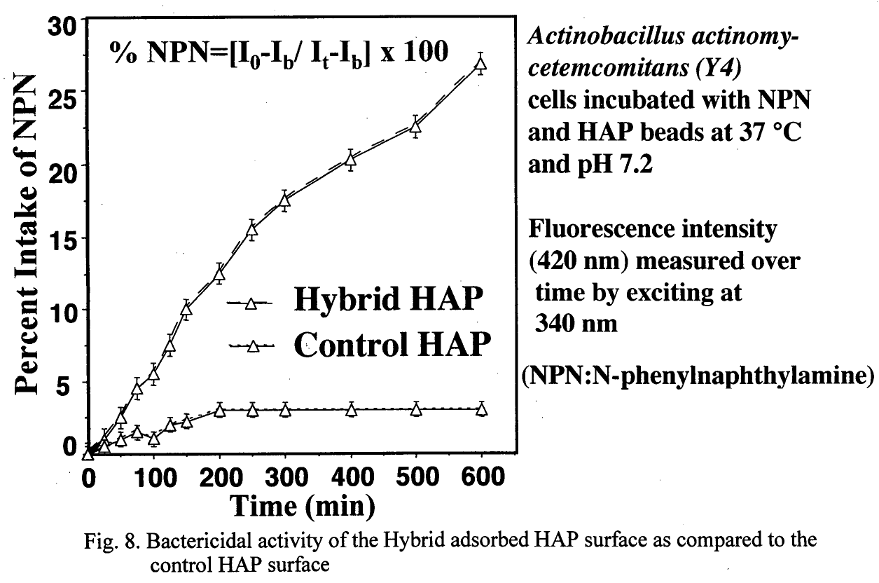

The fungicidal and bactericidal activity of the hybrid adsorbed HAP beads was determined using fluorescent probes. The increase in fluorescence as a function of microbial membrane damage was monitored. The data shown in FIGS. 7 and 8 demonstrate that the hybrid adsorbed HAP surface significantly caused microbial membrane damage as a function of time and release of the antimicrobial peptide when compared with the control HAP. Candida albicans and Actinobacillus actinomycetemcomitans were used as the fungal and bacterial organisms, respectively. The adsorption and affinity coefficient data are provided in Table 2 and FIG. 6.

TABLE 2
Adsorption Parameters for Peptides

| Peptide | K × 10⁻⁵ | N × 10⁶ |
|---|---|---|
| | (liter mol⁻¹) | (mol m⁻²) |
| Statherin | 11.1 ± 2.0 | 0.9 ± 0.05 |
| Carrier | 9.5 ± 1.5 | 2.1 ± 0.2 |
| Hybrid | 10.8 ± 1.8 | 1.6 ± 0.2 |

C/Q = 1/KN + C/N

TABLE 3
List of exemplary efficient carrier molecules

(SN15) [1-15]: D-Sp-Sp-E-E-K-F-L-R-R-I-G-R-F-G
Analog (SN15a): D-D-D-E-E-K-F-L-R-R-I-G-R-F-G
Analog (SN5): D-Sp-Sp-E-E
Analog (SN5a): D-D-D-E-E
(Salivary statherin fragments and their analogs;
Sp = Phosphoserine)
PRPN: X-D-L-D-E-D-V-S-Q-E-D-V-P-L-V-I-S-D-G-G-D-
S-E-Q-F-I-D-E-E-R,
wherein X is pyroglutamic acid.
(N-terminal fragment of salivary proline-rich protein)

TABLE 4
List of exemplary antimicrobial peptides

BN16: X-X-P-P-I-X-P-P-F-Y-P-P-F-X-P-P
[X=R, BN16; X=K, BN16a, X=ornithine, BN16b; X=D-arginine, BN16c]
(Fragments of Bactenecins)

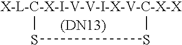

DN13 (X=R); DN13a (X=K) DN13_{b} (X=ornithine) DN13ₒ (X=D-arginine)
(Fragments of Defensins)
DN12: R-L-C-R-I-V-V-I-R-V-C-R
(Defensin-like Peptide from Bovine Neutrophils)

### References

Amano, A., Sojar, H. T., Lee, J. Y., Sharma, A., Levine, M. J. and Genco, R. J. Salivary receptors for recombinant fimbrillin of Porphyromonas gingivalis. Infect. Immun. 62:3372-3380, 1994.

Amano, A., Sharma, A., Lee, J. Y., Sojar, H. T., Raj, P. A. and Genco, R. J. Structural domains of Porphyromonas gingivalis recombinant fimbrillin that mediate binding to salivary proline-rich protein and statherin. Infect. Immun. 64:1631-1637, 1996.

Amano, A., Kataoka, K., Raj, P. A., Genco, R. J. and Shizukuishi, S. Binding sites of salivary statherin for Porphyromonas gingivalis recombinant fimbrillin. Infect. Immun. 64:4249-4254, 1996a.

Antonyraj, K. J., Karunakaran, T. and Raj, P. A. Bactericidal activity and poly-L-proline II conformation of the tandem repeat sequence of human salivary mucin glycoprotein (MG2). Arch. Biochem. Biophys. 356:197-208, 1998.

Darveau, R. P., Belton, C. M., Reife, R. A. and Lamont, R. J. Local chemokine paralysis, a novel pathogenic mechanism for Phorphyromonas gingivalis. Infect. Immun. 66:1660-1665, 1998.

Gibbons, R. J. and Hay, D. I. Human salivary acidic proline-rich proteins and statherin promote the attachment of Actinomyces viscosus LY7 to apatitic surfaces. Infec. Immun. 56:439-445,1988.

Gibbons, R. J., Hay, D. I. and. Schlesinger, D. H. Delineation of a segment of adsorbed salivary acidic proline-rich proteins which promotes adhesion of Streptococcus gordonii to apatitic surfaces. Infect Immun 59(9):2948-2954, 1991.

Goldman, M. J., Anderson, G. M., Stolenberg, E. D., Kari, U. P., Zasloff, M. and Wilson, J. M. Human β-defensin-1 is a self-sensitive antibiotic in lung that is inactivated in cystic fibrosis. Cell 88:553-560, 1997

Greenstein, G. and Polson, A. The role of local drug delivery in the management of periodontal diseases: a comprehensive review. J. Periodontol. 69:507-520, 1998.

Krisanaprakornkit, S., Weinberg, A., Perez, C. N. and Dale, B. A. Expression of the peptide antibiotic, human β-defensin I (hBD-1), in cultured human gingival epithelial cells and gingival tissues. Infect. Immun. 66:4222-4228, 1998.

Miyasaki, K. T. and Lehrer, R. I. Beta-sheet antibiotic peptide as potential dental therapeutics. Int. J. Antimicrob. Agents 9:269-280, 1998.

Raj, P. A., Johnsson, M., Levine, M. J. and Nancollas, G. H. Salivary statherin: dependence of charge, sequence, hydrophobicity, hydrogen bonding potency and helical conformation for adsorption to hydroxyapatite and inhibition of mineralization. J. Biol. Chem. 267:5968-5976, 1992.

Raj, P. A. and Edgerton, M. Functional Domain and Poly-L-proline II conformation for candidacidal activity of bactenecin 5. FEBS Lett. 368:526-530, 1995.

Raj, P. A., Marcus, E. and Edgerton, M. Delineation of an active fragment and poly (L-proline) II conformation for candidacidal activity of bactenecin 5. Biochemistry, 35:4314-4325, 1996.

Raj, P. A. and Genco, R. J. New and novel peptide antibiotics to overcome microbial resistance to conventional antibiotics. Impact of bacterial antibiotic resistance to oral health: What is the relevance? School of Public Health and Community Medicine, University of Washington, Seattle, 1996.

Raj, P. A., Gauri and Edgerton, M. Bactenecins: potent peptide antibiotics for Porphyromonas gingivalis and Actinobacillus actinomycetemcomitans. Int. J. Oral Biol. 22:73-80, 1997.

Raj, P. A., Antonyraj, K. J. and Karunakaran, T. Large scale synthesis and functional elements for antimicrobial activity of defensins. Biochem. J. 347:633-641, 2000.

Raj, P. A., Karunakaran, T. and Sukumaran, D. K. Synthesis, microbicidal activity and solution structure of a dodecapeptide sequence from bovine neutrophils. Biopolymers 53:281-292, 2000a.

Raj, P. A. and Antonyraj, K. J. Salivary statherin fragment elicit broad-spectrum antimicrobial activity, J. Dent. Res. 80:629 (Abst. # 822), 2001.

Selsted, M. E. and Quellette, A. J. Defensins in granules of phagocytic and non-phagocytic cells. Trends Cell Biol. 5:114-119, 1995.

Smith, J. J. Travis, S. M., Greenberg, E. P. and Welsh, M. J. Cystic fibrosis airway epithelia fail to kill because of abnormal airway surface fluid. Cell 85:229-239, 1996.

Weinberg, A., Krisanaprokornkit, S. and Dale, B. A. Epithelial antimicrobial peptides: Review and significance for oral applications. Crit. Rev. Oral Biol. Med. 9:399-414, 1998.

We claim:

1. A bifunctional protein molecule comprising at least two segments, wherein the first segment comprises a tooth enamel binding region of a salivary protein and wherein the second segment comprises an antimicrobial domain of a naturally occurring antimicrobial polypeptide, wherein the first segment and the second segment are linked by a biodegradable bond.

2. The molecule of claim 1 wherein the first segment is selected from the salivary protein selected from the group consisting of mucin, α-amylase, protein-rich glycoprotein, proline-rich proteins, cystatin, statherin, and histatins.

3. The molecule of claim 2 wherein the first segment is selected from a statherin.

4. The molecule of claim 3 wherein the first segment is selected from the statherin amino terminal 15 residue fragment (SN15) or its analog (SN_{A}15).

5. The molecule of claim 1 wherein the second segment is selected from a naturally occurring antimicrobial polypeptide selected from the group consisting of defensins, histatins, and bactenecins.

6. The molecule of claim 5 wherein the second segment is selected from a fragment of defensin with 12 to 16 residues containing one disulfide bridge.

7. The molecule of claim 5 wherein the second segment is selected from the BN16 bactenecin fragment, DN13, DN13a, DN13b, DN13₀ defensin fragment, DN12 defensin-like peptide from bovine neutrophil, bacterial-membrane permeability increasing proteins, and biofilm signaling molecules.

8. The method of claim 1 wherein the biodegradable bond is selected from the group consisting of peroxides, anhydrides, esters, ethers, and disulfides.

9. The method of claim 8 wherein the biodegradable bond is designed to provide slow delivery of a therapeutic function and is selected from the group consisting of esters and ethers.

10. The method of claim 1 wherein the biodegradable bond is designed to provide a short term and rapid release and is selected from the group consisting of anhydride and peroxide linkages.

11. The method of claim 8 wherein the biodegradable bond is selected from disulfide linkages and is designed to provide specific rate of release of antimicrobial agents in the presence of thiol-producing oral anaerobic bacterial pathogens.

12. A method of intra-orally delivering antimicrobial agents to tooth and pellicle surfaces, comprising the step of administering an effective amount of the molecule of claim 1 to the tooth or pellicle surface.

13. The method of claim 12 wherein the antimicrobial agent is delivered within a mouth rinse, toothpaste or gum.

14. The method of claim 12 wherein the antimicrobial agent is delivered by subgingival irrigation.

15. A bifunctional protein molecule comprising at least two segments wherein the first segment comprises a tooth enamel binding region of a salivary protein and wherein the second segment comprises a therapeutic molecule selected from the group consisting of anti-inflammatory agents, immuno-modulators, growth factors, and biofilm signalling agents and inhibitors; wherein the first and second segments are linked by a biodegradable bond.
